(19) United States
(12) Patent Application Publication
(10) Pub. No.: US 2024/0153595 A1
(43) Pub. Date: May 9, 2024

(54) SYSTEM AND METHOD FOR PERFORMING ACCELERATED MOLECULAR DYNAMICS COMPUTER SIMULATIONS WITH UNCERTAINTY-AWARE NEURAL NETWORK

(71) Applicant: QPIVOLTA TECHNOLOGIES PRIVATE LIMITED, BENGALURU (IN)

(72) Inventors: NAWAF ALAMPARA, PALAKKAD (IN); ASWANTH KRISHNAN, PALAKKAD (IN); NAGENDRA NAGARAJA, BENGALURU (IN)

(21) Appl. No.: 18/502,852

(22) Filed: Nov. 6, 2023

(30) Foreign Application Priority Data
Nov. 6, 2022 (IN) 202241050913

(51) Int. Cl. G16C 10/00; G06F 30/27; G16C 20/70

(52) U.S. Cl. CPC G16C 10/00; G06F 30/27; G16C 20/70

(57) ABSTRACT

The embodiments herein provide a system and method for performing accelerated molecular dynamics computer simulations with uncertainty-aware neural networks. The embodiments herein utilize a computational method to simulate the dynamics of atoms in a multi-element system using accelerated molecular dynamics using neural networks (NN) without compromising the accuracy. The formulated method involves simulating the system using ab initio molecular dynamics (AIMD) for a certain number of steps, which are utilized, to train the NN. Further, the trained NN can infer the further steps of the simulation. Here, the uncertainty of the prediction is closely monitored by incorporating uncertainty quantification into NN models. Uncertainty over the threshold indicates the need for more training and hence the usage of AIMD for a few more steps. Therefore, the embodiments herein help in delivering an accurate simulation results at an accelerated speed.

### CROSS-REFERENCE TO RELATED APPLICATIONS

The present application claims the priority of the Indian Provisional Patent Application (PPA) with serial number 202241050913 filed on Sep. 6, 2022 and subsequently postdated by 2 months to Nov. 2, 2023 with the title “A SYSTEM AND METHOD FOR PERFORMING ACCELERATED MOLECULAR DYNAMICS COMPUTER SIMULATIONS WITH UNCERTAINTY-AWARE NEURAL NETWORK”. The contents of the abovementioned Applications are included in entirety as reference herein.

### BACKGROUND

### Technical Field

The embodiments herein generally relate to the field of artificial intelligence. The embodiments herein are particularly related to neural network force field (NNFF) computational algorithms for direct prediction of atomic forces in molecular dynamics computer simulations in material systems. The embodiments herein are more particularly related to a system and method to couple uncertainty-monitored NNs (Neural Networks) with molecular dynamics for directly predicting the energy of the system and forces acting on atoms in molecular dynamic simulation of materials, polymers, and molecules systems with application in, but not limited to electrochemical, photoelectrochemical and semiconductor devices.

### Description of the Related Art

The discovery of materials with various functional applications ranging from catalysis to energy storage to electronics is the key factor toward a transition in the technology. Conventional trial-and-error experimental approaches to selecting materials from combinatorically huge material space require decades of research and large financial investments.

Methods like molecular dynamics and DFT modeling have gained traction in computational methods to investigate material properties. The application of the computationally studied material includes but is not limited to, battery materials, energy storage technology, catalysis, fuel cells, photovoltaics, thermos-electrics, energy conversion, sensing, carbon capture, and so on.

Moreover, molecular dynamics is one of the various computational methodologies to simulate the dynamics of atoms under various conditions in various systems, including materials, molecules, polymers, liquids, and so on. Molecular dynamics simulations help to simulate and probe phenomena that are experimentally not yet possible or difficult to study. The dynamics of the atoms are studied, where the forces acting on the system and the energy of the system can be calculated using various approaches. AIMD is a molecular dynamics method based on first principles. First principle approaches based on quantum mechanics (Schrödinger and Kohn-Sham equations) can be used to calculate the interatomic forces by solving many-body equations. However, the computational expense of AIMD limits the usage of this method to very small systems (fewer atoms) and for smaller durations (fewer time steps). Furthermore, physical phenomena, if they have to be simulated realistically, require a sufficiently large system (many numbers of atoms) and for longer durations (many time steps).

Therefore, AIMD simulations, since they don't make any prior assumption on the potential energy surface and are calculated based on QM, can accurately simulate many physical phenomena. Classical molecular dynamics (CMD) methods, however, use pre-fitted empirical functional forms based on prior experiments, calculations, and assumptions and are valid only in certain conditions. These pre-fitted potentials cannot be used to study complex interactions depending on the functional forms. However, there are more complex pre-defined functionals, such as ReaxFF, for classical molecular dynamics, which can be used to simulate chemical reactions, but parametrizing these functionals is very challenging. Compared to AIMD, since the interatomic forces were fitted into pre-defined functional forms, this allows acceleration of simulation by several orders of magnitude and can be used to study amorphous systems, polymers, interfaces, and nanostructures.

However, due to the advancements in NN models and the availability of large DFT datasets, machine learning (ML) has quickly gained traction as a powerful and efficient tool to accelerate quantum simulations and, in some cases, even as an alternative tool. The data-driven approach has the potential to reduce the computation-experiment cycle time in the conventional approach and can accelerate material discovery. The data-driven approach gives us a very large speedup in terms of computing, which further will allow us to study a larger number of materials in a high throughput fashion in a reasonable time. The application of these models has enormous potential to develop new technologies in the industry and further the fundamental understanding of science.

Hence, in the view of this, there is need for a system and method to perform accelerated molecular dynamics computer simulations with uncertainty-aware neural network

The above-mentioned shortcomings, disadvantages and problems are addressed herein, and which will be understood by reading and studying the following specification.

OBJECTIVES OF THE EMBODIMENTS HEREIN

The primary object of the embodiments herein is to provide a system and method for performing accelerated molecular dynamics computer simulations with uncertainty-aware neural networks.

Another object of the embodiments herein is to provide a computational method to simulate the dynamics of atoms in a multi-element system using accelerated molecular dynamics using neural networks (NN) without compromising accuracy.

Yet another object of the embodiments herein is to provide a method involving simulating the system using ab initio molecular dynamics (AIMD) for a certain number of steps, which are utilized, to train the neural network.

Yet another object of the embodiments herein is to provide a method providing trained NN, that provides further steps of simulation accurately at an accelerated speed.

Yet another object of the embodiments herein is to provide a method for performing accelerated molecular dynamics computer simulations with uncertainty-aware neural networks, such that the uncertainty of the prediction is closely monitored by incorporating uncertainty quantification into NN models.

Yet another object of the embodiments herein is to provide a method for performing accelerated molecular dynamics computer simulations with an uncertainty-aware neural network, wherein uncertainty over the user-specified threshold indicates the need for more training and hence the usage of AIMD for a few more steps.

These and other objects and advantages of the present invention will become readily apparent from the following detailed description taken in conjunction with the accompanying drawings.

### SUMMARY

The following details present a simplified summary of the embodiments herein to provide a basic understanding of the several aspects of the embodiments herein. This summary is not an extensive overview of the embodiments herein. It is not intended to identify key/critical elements of the embodiments herein or to delineate the scope of the embodiments herein. Its sole purpose is to present the concepts of the embodiments herein in a simplified form as a prelude to the more detailed description that is presented later.

The other objects and advantages of the embodiments herein will become readily apparent from the following description taken in conjunction with the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

This Summary is provided to introduce a selection of concepts in a simplified form that is further described below in the Detailed Description. This Summary is not intended to identify key or essential features of the claimed subject matter, nor is it intended to be used as an aid in determining the scope of the claimed subject matter.

The various embodiments herein provide a system and method for performing accelerated molecular dynamics computer simulations with uncertainty-aware neural networks. The embodiments herein couples Neural Networks (NNs) with Ab initio molecular dynamics (AIMD) for on-the-fly training and prediction of interatomic potentials. Forces are then computed by taking the energy gradient with respect to positions. The NN prediction can accelerate the quantum simulation by directly predicting the energy instead of self-consistent quantum calculation. Furthermore, the NN model would be trained for the initial few frames of the trajectories on the fly from AIMD calculation. Subsequent frames of the trajectories would be predicted by the NN. Then, the trained model would be used to run multiple AIMD simulations with random initialization for certainty. Training the model on each material would help the model generalize to that specific material and learn the atom embedding from lesser data.

According to one embodiment herein, a method for performing accelerated molecular dynamics computer simulations with an uncertainty-aware neural network is provided. The method comprises receiving an input request to initialize simulations, comprising initial material 3D structure or compositions, and metadata comprising parameters relevant for MD or molecular dynamics simulations. The method further involves running the molecular dynamics simulations based on the input request, to predict the simulation trajectory of movement of atoms, comprising energy of the system and forces acting on the atoms as simulation, and storing the simulation trajectories in a data store. In addition, the method involves training a neural network force field NNFF using the simulation trajectories stored in the data store and storing the trained NNFF parameters in a model store. Furthermore, the method comprises running the molecular dynamics simulations with the trained NNFF parameters stored in the model store to predict the further steps of simulation trajectory movement of atoms, comprising energy of the system and forces acting on the atoms as simulation. In addition, the method involves monitoring uncertainty while predicting further simulation trajectory of movement of atoms by the trained NNFF, launching MD simulations, and replenishing the data store for training or retraining the NNFF, in case of detecting uncertainty over the threshold. Furthermore, the method involves calculating material properties by post-processing the MD simulation trajectory data.

According to one embodiment herein, the input request to initialize simulations comprises atom types, a 3D structure including coordinates, lattice parameters, the composition of the system, and the velocity of the atoms, such that the velocities may be randomly assigned according to a desired temperature or obtained from a previous equilibration run.

According to one embodiment herein, the metadata parameters comprise the values corresponding to ensembles, including NVT, number of particles (N), volume (V), temperature (T), NPT, number of particles (N), pressure (P), temperature (T), and NVE, number of particles (N), volume (V), energy (E). Integration algorithm to numerically solve the equations of motion for the particles in the system, such as velocity verlet algorithms.

According to one embodiment herein, the molecular dynamics simulations based on the input request, to predict the simulation trajectory of the movement of atoms are run for a number of timesteps by MD worker nodes. The number of timesteps depends upon the complexity of the system, including large and small systems, such that the large system involves more interactions and longer equilibrium times, and the small system involves fewer interactions and shorter equilibrium times.

According to one embodiment herein, the method for training neural network force field NNFF is provided. The method includes using simulation trajectories stored in the datastore generated based on MD simulations. The simulation trajectories include snapshots of the system at different timesteps. The method further includes normalizing and preprocessing the simulation trajectories. In addition, the method involves defining a loss function that quantifies the error between the predicted future steps and the actual future steps in the simulation. Common loss functions include mean squared error (MSE) or a physics-based loss function that considers energy conservation and physical constraints. Furthermore, training the NNFF using the prepared simulation trajectories and the loss function.

According to one embodiment herein, the neural network force field NNFF is trained using the simulation trajectories stored in the data store for a number of iterations by NN trainer nodes. The number of iterations is a hyperparameter, which is set to a default value by a user and changed accordingly depending on the system needs to be simulated.

According to one embodiment herein, the uncertainty is monitored by an uncertainty monitoring service while running further MD with the NNFF. The uncertainty monitoring service if it detects uncertainty above prescribed threshold then method involves launching MD simulations and replenishing the data store for training or retraining the NNFF. The training or retraining involves fine-tuning with new data or adding more data and training the NNFF module again with the inputs from molecular dynamics.

According to one embodiment herein, the post-processing of MD simulation trajectory data involves analyzing and extracting various properties and insights from the simulated atomic trajectories, and the properties provide valuable information about the behavior of the system under study. The properties include structural properties, thermodynamic properties, dynamics and kinetics, chemical properties, hydration properties, electrostatic and non-bonded interactions, conformational analysis, hydrogen bond analysis, energetics, and other associated properties.

According to one embodiment herein, a system for performing accelerated molecular dynamics computer simulations with an uncertainty-aware neural network is provided. The system comprises an input module configured to receive an input request to initialize simulations, comprising initial material 3D structure or compositions, metadata comprising of parameters relevant for MD or molecular dynamics simulations. The system further comprises a molecular dynamics (MD) module configured to receive the input request from the input module and run the molecular dynamics simulations based on the input request, to predict the simulation trajectory of movement of atoms, comprising energy of the system and forces acting on the atoms as simulation, and storing the simulation trajectories in a data store. In addition, the system comprises a neural network force field NNFF module configured to receive the simulation trajectory of movement of atoms from the MD module and train the network force field NNFF module using the simulation trajectories stored in the data store, and store the trained NNFF parameters in a model store. The NNFF module is further configured to run the molecular dynamics simulations with the trained NNFF parameters stored in the model store to predict the further steps of simulation trajectory movement of atoms, comprising energy of the system and forces acting on the atoms as simulation. Furthermore, the system comprises an uncertainty monitoring service module configured to monitor uncertainty involved while predicting further simulation trajectory of movement of atoms by the NNFF module, and also configured to request more data from the MD module to replenish the datastore for training or retraining the NNFF module again in case of uncertainty over the threshold, and to calculate material properties by post-processing the MD simulation trajectory data.

According to one embodiment herein, the input request by the input module to initialize simulations comprises atom types, a 3D structure including coordinates, lattice parameters, the composition of the system, and the velocity of the atoms.

According to one embodiment herein, the metadata parameters comprise the values corresponding to ensembles, including NVT, number of particles (N), volume (V), temperature (T), NPT, number of particles (N), pressure (P), temperature (T), and NVE, number of particles (N), volume (V), energy (E), integration algorithm to numerically solve the equations of motion, timestep to determine the frequency of integration of equations of motion, boundary conditions, and cutoff distance.

According to one embodiment herein, the molecular dynamics simulations based on the input request by the MD module, to predict the simulation trajectory of movement of atoms is run for a number of timesteps by MD worker nodes. The number of timesteps depend upon the complexity of the system, including large and small system, and wherein the large system involves more interactions and longer equilibrium times, and the small system involves fewer interactions and shorter equilibrium times.

According to one embodiment herein, the method for training neural network force field NNFF in the NNFF module comprises the steps of: using the simulation trajectories stored in the datastore generated based on MD simulations. The simulation trajectories include snapshots of the system at different timesteps. The method further comprises normalizing and preprocessing the simulation trajectories. In addition, define the loss function that quantifies the error between the predicted future steps and the actual future steps in the simulation, and training the NNFF using the prepared simulation trajectories and the loss function.

According to one embodiment herein, the neural network force field NNFF module is trained using the simulation trajectories stored in the data store for a number of iterations by NN trainer nodes. The number of iterations is a hyperparameter, which is set to a default value by a user and changed accordingly depending on the system needs to be simulated.

According to one embodiment herein, the uncertainty monitoring service module is configured to monitor uncertainty by incorporating uncertainty quantification into the NNFF module, provided if the uncertainty detected is above the prescribed threshold, then the uncertainty monitoring service module is configured to requests more data from the MD module and replenishing the data store for training or retraining the NNFF. The training or retraining involves fine-tuning with new data or adding more data and training the NNFF module again with the inputs from the molecular dynamics module. The uncertainty quantification helps in making more informed decisions and assessing the reliability of the model predictions.

According to one embodiment herein, the post-processing of MD simulation trajectory data involves analyzing and extracting various properties and insights from the simulated atomic trajectories, and the properties provide valuable information about the behavior of the system under study; and wherein the properties include structural properties, thermodynamic properties, dynamics and kinetics, chemical properties, hydration properties, electrostatic and non-bonded interactions, conformational analysis, hydrogen bond analysis, energetics, and other associated properties.

The foregoing summary is illustrative only and is not intended to be in any way limiting. In addition to the illustrative aspects, embodiments, and features described above, further aspects, embodiments, and features will become apparent by reference to the drawings and the following detailed description.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow-diagram of a method for performing accelerated molecular dynamics computer simulations with an uncertainty-aware neural network, according to an embodiment herein.

FIG. 2 illustrates a workflow of a system for performing accelerated molecular dynamics computer simulations with uncertainty-aware neural networks, according to an embodiment herein.

FIG. 3 illustrates a schematic representation of AIMD run (data generation and training) and inference of the uncertainty monitoring cycle, in accordance with an embodiment herein.

FIG. 4 illustrates a schematic representation of uncertainty monitoring, in accordance with an embodiment herein.

FIG. 5A illustrates an architecture of an uncertainty monitoring service, in accordance with an embodiment herein.

FIG. 5B illustrates a schematic workflow for uncertainty monitoring service, in accordance with an embodiment herein.

FIG. 6 illustrates a schematic diagram of a computing platform utilized to implement NNFF algorithm, in accordance with an embodiment herein.

FIG. 7 illustrates depicts graph showing results for the prediction error in forces during molecular simulation of lithium thiophosphate (LiPS) system, in accordance with an embodiment herein.

FIG. 8 illustrates a screenshot of the user interface for performing molecular dynamics simulations of catalyst using NNFF algorithm, in accordance with an embodiment herein.

Although the specific features of the present invention are shown in some drawings and not in others. This is done for convenience only as each feature may be combined with any or all of the other features in accordance with the present invention.

### DETAILED DESCRIPTION OF THE EMBODIMENTS HEREIN

In the following detailed description, reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical, and other changes may be made without departing from the scope of the embodiments. The following detailed description is therefore not to be taken in a limiting sense.

The foregoing of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings. Although the terms first, second, etc. may be used herein to describe various elements, these elements should not be limited by these terms. These terms are generally only used to distinguish one element from another.

The various embodiments herein provide a system and method for performing accelerated molecular dynamics computer simulations with uncertainty-aware neural networks. The embodiments herein couples Neural Networks (NNs) with Ab initio molecular dynamics (AIMD) for on-the-fly training and prediction of interatomic potentials. Forces are then computed by taking the energy gradient with respect to positions. The NN prediction can accelerate the quantum simulation by directly predicting the energy instead of self-consistent quantum calculation. Furthermore, the NN model would be trained for the initial few frames of the trajectories on the fly from AIMD calculation. Subsequent frames of the trajectories would be predicted by the NN. Then, the trained model would be used to run multiple AIMD simulations with random initialization for certainty. Training the model on each material would help the model generalize to that specific material and learn the atom embedding from lesser data.

According to one embodiment herein, a method for performing accelerated molecular dynamics computer simulations with an uncertainty-aware neural network is provided. The method comprises receiving an input request to initialize simulations, comprising initial material 3D structure or compositions, and metadata comprising parameters relevant for MD or molecular dynamics simulations. The method further involves running the molecular dynamics simulations based on the input request, to predict the simulation trajectory of movement of atoms, comprising energy of the system and forces acting on the atoms as simulation, and storing the simulation trajectories in a data store. In addition, the method involves training a neural network force field NNFF using the simulation trajectories stored in the data store and storing the trained NNFF parameters in a model store. Furthermore, the method comprises running the molecular dynamics simulations with the trained NNFF parameters stored in the model store to predict the further steps of simulation trajectory movement of atoms, comprising energy of the system and forces acting on the atoms as simulation. In addition, the method involves monitoring uncertainty while predicting further simulation trajectory of movement of atoms by the trained NNFF, launching MD simulations, and replenishing the datastore for training or retraining the NNFF, in case of detecting uncertainty over the threshold. Furthermore, the method involves calculating material properties by post-processing the MD simulation trajectory data.

According to one embodiment herein, the input request to initialize simulations comprises atom types, a 3D structure including coordinates, lattice parameters, the composition of the system, and the velocity of the atoms, such that the velocities may be randomly assigned according to a desired temperature or obtained from a previous equilibration run.

According to one embodiment herein, the metadata parameters comprise the values corresponding to ensembles, including NVT, number of particles (N), volume (V), temperature (T), NPT, number of particles (N), pressure (P), temperature (T), and NVE, number of particles (N), volume (V), energy (E). Integration algorithm to numerically solve the equations of motion for the particles in the system, such as velocity verlet algorithms. Furthermore, the metadata parameter includes timestep to determine the frequency of integration of equations of motion. A typical value might be 1 femtosecond (fs) or smaller, and simulation length which is the total simulation time. In addition, the metadata parameters include boundary conditions, and cutoff distance for non-bonded interactions, such as van der Waals and electrostatic. Besides atomic positions and velocities are also included, and also the data collected during the simulation, such as trajectories, energies, radial distribution functions, and various structural properties.

According to one embodiment herein, the molecular dynamics simulations based on the input request, to predict the simulation trajectory of the movement of atoms are run for a number of timesteps by MD worker nodes. The number of timesteps depends upon the complexity of the system, including large and small systems, such that the large system involves more interactions and longer equilibrium times, and the small system involves fewer interactions and shorter equilibrium times.

Hence, MD simulations often require an equilibration phase to relax the system to its desired state. The duration of this equilibration phase depends on the system's complexity and the desired properties to be sampled during production. To obtain statistically significant results, simulations must cover a sufficient number of independent samples. The number of samples needed depends on the property being studied and can be estimated through statistical analysis.

MD helps in numerically solving the equations of motion for all the atoms in the system. The MD simulation begins with an initial configuration of atoms, including their positions and velocities. These initial conditions can be based on experimental data or generated by other means. The positions specify where each atom is located within a simulation box, and the velocities indicate their initial speeds and directions.

Furthermore, the force field is used to calculate the forces acting on each atom in the system. The force field includes mathematical equations and parameters that describe the interactions between atoms, such as bonded interactions (bonds, angles, dihedrals) and non-bonded interactions (van der Waals forces and electrostatic forces). The force on each atom is computed based on the positions of neighboring atoms and the force field equations. In the case of Ab Initio Molecular Dynamics, the force field is based on Density functional Theory (DFT) which is a quantum mechanical approximation to compute the energy of a system.

In addition, to predict the positions and velocities of atoms changing over time, an integration algorithm is applied. The most commonly used algorithm is the Verlet algorithm, which numerically integrates the equations of motion, such as Newton's second law over small time steps (Δt). The algorithm calculates new positions and velocities for each atom in discrete time steps. The simulation proceeds by repeatedly advancing time in discrete steps of Δt. During each step force, velocity, and position is calculated. The force is calculated based on the current positions of atoms, the forces acting on each atom using the force field parameters. Further, velocity is calculated using the calculated forces and the current velocities to update the velocities of all atoms. In addition, the position of all the atoms is calculated based on their new velocities and the current positions.

According to one embodiment herein, the method for training neural network force field NNFF is provided. The method includes using simulation trajectories stored in the datastore generated based on MD simulations. The simulation trajectories include snapshots of the system at different timesteps. The method further includes normalizing and preprocessing the simulation trajectories. In addition, the method involves defining a loss function that quantifies the error between the predicted future steps and the actual future steps in the simulation. Common loss functions include mean squared error (MSE) or a physics-based loss function that considers energy conservation and physical constraints. Furthermore, training the NNFF using the prepared simulation trajectories and the loss function.

Moreover, standard optimization techniques, such as stochastic gradient descent (SGD) or more advanced optimizers like Adam can be used. This may involve collecting more data, adjusting the architecture, or fine-tuning hyperparameters. Once trained, a neural network model can be deployed to predict future steps of your simulation. The trained neural network force field model (NNFF) can then act as an alternative to the forcefield in MD, where it can be used to compute energy and forces. The inputs for predictions would be the same to that of the MD inputs. Based on the uncertainty estimates on the subsequent predictions, iteration is carried out if required.

According to one embodiment herein, the neural network force field NNFF is trained using the simulation trajectories stored in the data store for a number of iterations by NN trainer nodes. The number of iterations is a hyperparameter, which is set to a default value by a user and changed accordingly depending on the system needs to be simulated.

According to one embodiment herein, the uncertainty is monitored by an uncertainty monitoring service while running further MD with the NNFF. The uncertainty monitoring service if it detects uncertainty above the prescribed threshold then the method involves launching MD simulations and replenishing the data store for training or retraining the NNFF. The training or retraining involves fine-tuning with new data or adding more data and training the NNFF module again with the inputs from molecular dynamics.

There are various methods incorporated while monitoring the uncertainty, which accounts for the prediction of the uncertainty, such as Bayesian NN (BNN), Deep Ensemble, and Monte Carlo (MC) dropout. The Bayesian NN (BNN) is one of the most popular methods for quantifying uncertainty by inferring predictive distribution. The variance in the predictive distribution is used to measure the uncertainty. Therefore, instead of having a single set of weights, weights and outputs are treated as probabilistic variables and we try to find the marginal distributions that best fit the data. Furthermore, the BNN would find the distributions of the weights by catering to the probability distributions.

In addition, the Deep ensemble is yet another powerful approach to measuring uncertainty, where many copies of the model itself are trained on the same dataset. During inference, the average of the predictions or a combined output is treated as the prediction. Therefore, the variance or the distribution of the predictions is used to measure the uncertainty.

Furthermore, the Monte Carlo dropout is yet another method that is less compute-intensive to quantify uncertainty. MC dropout is a popular regularization technique to tackle over-fitting. Neurons are randomly deactivated, usually according to a Bernoulli distribution. In MC-dropout, randomly sampled random subnetworks are then trained on the same data. Furthermore, the MC dropouts are applied at both training and during inference, and the inference is done multiple times, with different parameters being dropped in each iteration. Finally, the outputs are combined typically by averaging over the number of iterations to output the probability. Based on the uncertainty estimation, training of the NNFF is carried out which can be used to predict subsequent steps of MD.

According to one embodiment herein, the post-processing of MD simulation trajectory data involves analyzing and extracting various properties and insights from the simulated atomic trajectories, and the properties provide valuable information about the behavior of the system under study. The properties include structural properties, thermodynamic properties, dynamics and kinetics, chemical properties, hydration properties, electrostatic and non-bonded interactions, conformational analysis, hydrogen bond analysis, energetics, and other associated properties.

The structural properties include the radial distribution function, which calculates the probability density of finding a particle at a certain distance from a reference particle, providing information about the density and arrangement of particles in the system. Pair Correlation Function which is similar to RDF but considers the relative positions of all particle pairs, provides a more detailed view of particle correlations.

The thermodynamic properties include temperature calculated using the kinetic energies of the particles, pressure, density, internal energy which is the sum of kinetic and potential energies, and entropy calculated from the velocity distribution or using statistical mechanics. The dynamics and kinetics including diffusion coefficients, to quantify the rate of diffusion of particles in the system. Mean Square Displacement (MSD) that measures the average squared displacement of particles over time. Further, viscosity can be estimated from the velocity autocorrelation function, and self-diffusion of individual particles.

The chemical properties including bond lengths and angles help to track the evolution of chemical bonds and angles over time, dihedral angles, and radical and chemical Reactions to detect and analyze chemical reactions or radical formation. Furthermore, the hydration properties include solvation structure to study the arrangement of solvent molecules around solute molecules, and hydrogen bond analysis to identify and quantify hydrogen bond interactions. Moreover, the electrostatic and non-bonded interactions, such as coulombic interactions to calculate the electrostatic energy and forces between charged particles. Van der Waals interactions to calculate Lennard-Jones potential energies and forces.

Furthermore, conformational analysis includes root mean square deviation to measure the deviation of molecular structures from a reference structure. The root mean square fluctuation to quantify the flexibility of individual atoms or residues. In addition, the hydrogen bond analysis includes hydrogen bond lifetimes to track the duration of hydrogen bond interactions, and hydrogen bond networks to analyze the connectivity of hydrogen bonds within the system. The energy includes potential energy, kinetic energy, and total energy. In addition, other relevant properties include Gyration Radius to measures the radius of gyration of a molecule or polymer, order parameters to quantify the degree of order or alignment of molecules, and diffusion coefficients to estimate the rate of diffusion for specific species.

According to one embodiment herein, a system for performing accelerated molecular dynamics computer simulations with an uncertainty-aware neural network is provided. The system comprises an input module configured to receive an input request to initialize simulations, comprising initial material 3D structure or compositions, and metadata comprising of parameters relevant for MD or molecular dynamics simulations. The system further comprises a molecular dynamics (MD) module configured to receive the input request from the input module and run the molecular dynamics simulations based on the input request, to predict the simulation trajectory of movement of atoms, comprising energy of the system and forces acting on the atoms as simulation, and storing the simulation trajectories in a data store. In addition, the system comprises a neural network force field NNFF module configured to receive the simulation trajectory of movement of atoms from the MD module and train the network force field NNFF module using the simulation trajectories stored in the data store, and store the trained NNFF parameters in a model store. The NNFF module is further configured to run the molecular dynamics simulations with the trained NNFF parameters stored in the model store to predict the further steps of simulation trajectory movement of atoms, comprising energy of the system and forces acting on the atoms as simulation. Furthermore, the system comprises an uncertainty monitoring service module configured to monitor uncertainty involved while predicting further simulation trajectory of movement of atoms by the NNFF module, and also configured to request more data from the MD module to replenish the datastore for training or retraining the NNFF module again in case of uncertainty over the threshold, and to calculate material properties by post-processing the MD simulation trajectory data.

According to one embodiment herein, the input request by the input module to initialize simulations comprises atom types, a 3D structure including coordinates, lattice parameters, the composition of the system, and the velocity of the atoms.

According to one embodiment herein, the metadata parameters comprise the values corresponding to ensembles, including NVT, number of particles (N), volume (V), temperature (T), NPT, number of particles (N), pressure (P), temperature (T), and NVE, number of particles (N), volume (V), energy (E), integration algorithm to numerically solve the equations of motion, timestep to determine the frequency of integration of equations of motion, boundary conditions, and cutoff distance.

According to one embodiment herein, the molecular dynamics simulations based on the input request by the MD module, to predict the simulation trajectory of movement of atoms is run for a number of timesteps by MD worker nodes. The number of timesteps depend upon the complexity of the system, including large and small system, and wherein the large system involves more interactions and longer equilibrium times, and the small system involves fewer interactions and shorter equilibrium times.

According to one embodiment herein, the method for training neural network force field NNFF in the NNFF module comprises the steps of: using the simulation trajectories stored in the datastore generated based on MD simulations. The simulation trajectories include snapshots of the system at different timesteps. The method further comprises normalizing and preprocessing the simulation trajectories. In addition, define the loss function that quantifies the error between the predicted future steps and the actual future steps in the simulation, and training the NNFF using the prepared simulation trajectories and the loss function.

According to one embodiment herein, the neural network force field NNFF module is trained using the simulation trajectories stored in the data store for a number of iterations by NN trainer nodes. The number of iterations is a hyperparameter, which is set to a default value by a user, and changed accordingly depending on the system needs to be simulated.

According to one embodiment herein, the uncertainty monitoring service module is configured to monitor uncertainty by incorporating uncertainty quantification into the NNFF module, provided if the uncertainty detected is above the prescribed threshold, then the uncertainty monitoring service module is configured to requests more data from the MD module and replenishing the data store for training or retraining the NNFF. The training or retraining involves fine-tuning with new data or adding more data and training the NNFF module again with the inputs from the molecular dynamics module. The uncertainty quantification helps in making more informed decisions and assessing the reliability of the model predictions.

According to one embodiment herein, the post-processing of MD simulation trajectory data involves analyzing and extracting various properties and insights from the simulated atomic trajectories, and the properties provide valuable information about the behavior of the system under study. The properties include structural properties, thermodynamic properties, dynamics and kinetics, chemical properties, hydration properties, electrostatic and non-bonded interactions, conformational analysis, hydrogen bond analysis, energetics, and other associated properties.

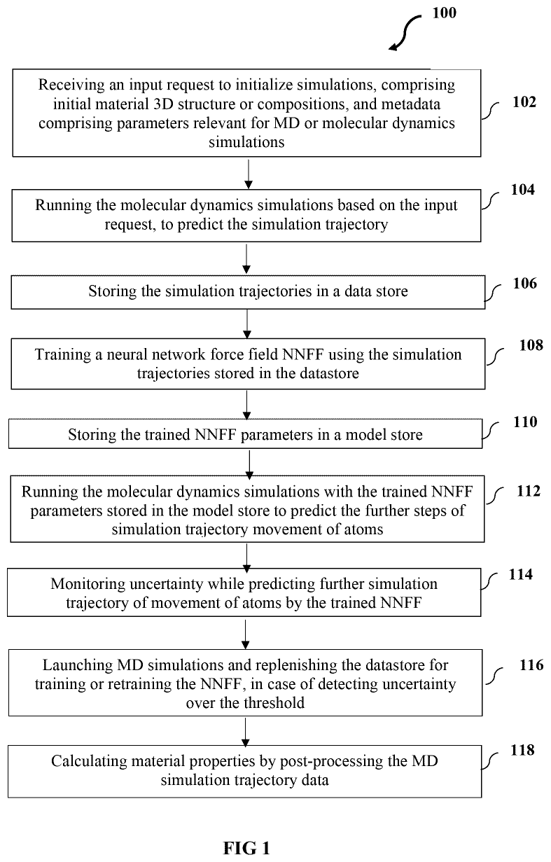

FIG. 1 illustrates a flow diagram of a method for performing accelerated molecular dynamics computer simulations with uncertainty-aware neural networks, according to an embodiment herein. The method 100 comprises receiving an input request to initialize simulations, comprising initial material 3D structure or compositions, and metadata comprising parameters relevant for MD or molecular dynamics simulations at step 102. The method further involves running the molecular dynamics simulations based on the input request, to predict the simulation trajectory of movement of atoms, comprising energy of the system and forces acting on the atoms as simulation at step 104, and storing the simulation trajectories in a data store at step 106. In addition, the method 100 involves training a neural network force field NNFF using the simulation trajectories stored in the datastore at step 108, and storing the trained NNFF parameters in a model store at step 110. Furthermore, the method 100 comprises running the molecular dynamics simulations with the trained NNFF parameters stored in the model store to predict the further steps of simulation trajectory movement of atoms, comprising energy of the system and forces acting on the atoms as simulation at step 112. In addition, the method 100 involves monitoring uncertainty while predicting further simulation trajectory of movement of atoms by the trained NNFF at step 114, and launching MD simulations and replenishing the datastore for training or retraining the NNFF, in case of detecting uncertainty over the threshold at step 116. Furthermore, the method 100 involves calculating material properties by post-processing the MD simulation trajectory data 118.

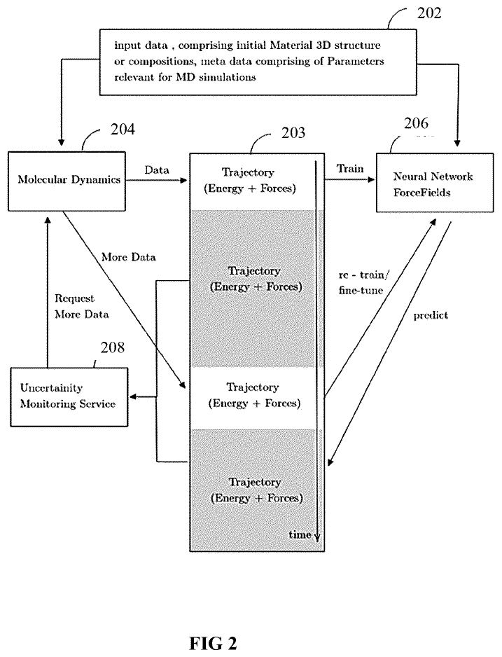

FIG. 2 illustrates an exemplary system for performing accelerated molecular dynamics computer simulations with an uncertainty-aware neural network, according to an embodiment herein. The system 200 comprises an input module 202 configured to receive an input request to initialize simulations, comprising initial material 3D structure or compositions, metadata comprising of parameters relevant for MD or molecular dynamics simulations. The system 200 further comprises a molecular dynamics (MD) module 204 configured to receive the input request from the input module and run the molecular dynamics simulations based on the input request, to predict the simulation trajectory of movement of atoms 203, comprising energy of the system and forces acting on the atoms as simulation, and storing the simulation trajectories in a data store. In addition, the system 200 comprises a neural network force field NNFF module 206 configured to receive the simulation trajectory of movement of atoms from the MD module and train the network force field NNFF module using the simulation trajectories stored in the data store, and store the trained NNFF parameters in a model store. The NNFF module 206 is further configured to run the molecular dynamics simulations with the trained NNFF parameters stored in the model store to predict the further steps of simulation trajectory movement of atoms, comprising energy of the system and forces acting on the atoms as simulation. Furthermore, the system 200 comprises an uncertainty monitoring service module 208 configured to monitor uncertainty involved while predicting further simulation trajectory of movement of atoms by the NNFF module, and also configured to request more data from the MD module to replenish the data store for training or retraining the NNFF module 206 again in case of uncertainty over the threshold, and to calculate material properties by post-processing the MD simulation trajectory data.

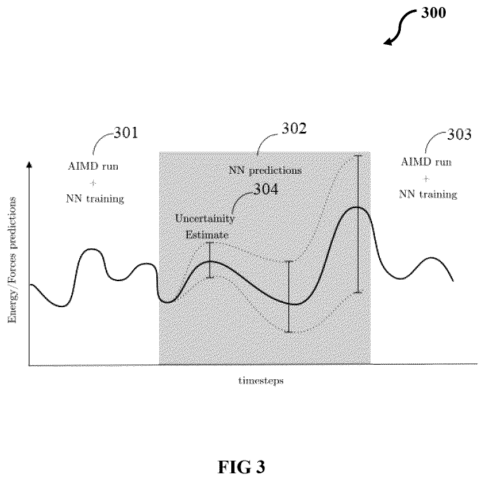

FIG. 3 illustrates a schematic representation of AIMD run (data generation and training) and inference of uncertainty monitoring cycle, according to an embodiment herein. The FIG. 3, 300 illustrates a schematic representation of energy/forces prediction vs timesteps comprising the AIMD run data generation and training 301, inference of uncertainty monitoring while NN predictions 302 and AIMD run data generation and training 303. The methodology 300 is continued until the desired timesteps are simulated. Furthermore, 304 indicates the estimation of uncertainty, during neural network NN predictions.

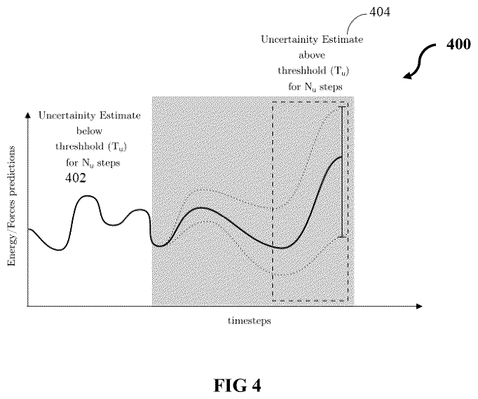

FIG. 4 illustrates a schematic representation of uncertainty monitoring, according to an embodiment herein. The FIG. 4, 400 illustrates estimation of uncertainty, during NN predictions of energy/forces. The width of the uncertainty estimate is used to monitor the uncertainty. 402 represents the uncertainty estimate below the user-specified threshold (Tᵤ) for (Nᵤ) steps. The Tᵤ is the threshold set for uncertainty, and Nᵤ is the allowed number of uncertain steps or subsequent predictions with an uncertainty value above Tᵤ. Furthermore, predictions with uncertainty above Tᵤ are not allowed after Nᵤ is crossed. 404 represents NN model prediction with uncertainty above the user-specified threshold (Tᵤ) for (Nᵤ) steps, wherein retraining of the NN model is required in case of uncertainty prediction is above the user-specified threshold.

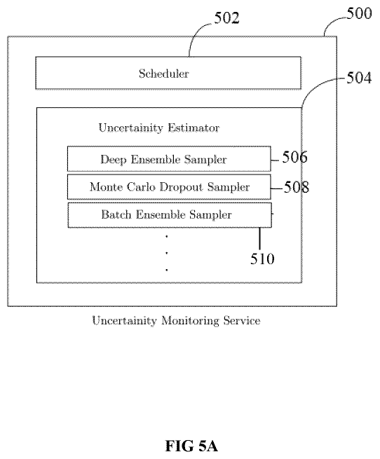

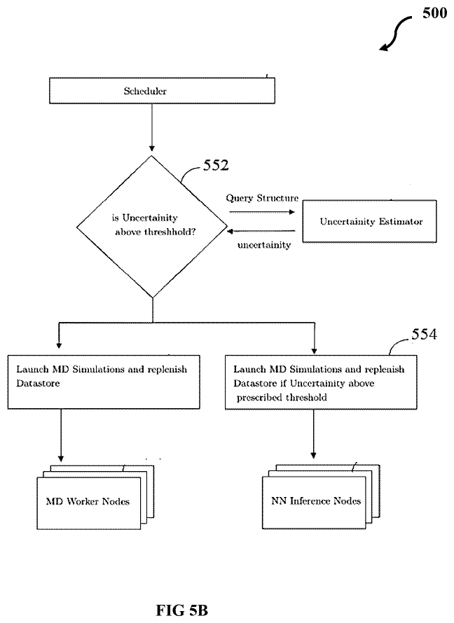

FIG. 5A illustrates an architecture of an uncertainty monitoring service, according to an embodiment herein. The FIG. 5A, 500 illustrates the architecture of the uncertainty monitoring service, comprising a scheduler 502 and an uncertainty estimator 504. The scheduler 502 schedules training and running of molecular dynamics based on uncertainty monitoring results, and the uncertainty estimator 504 estimates the uncertainty of the predictions. The uncertainty estimator further comprises Deep Ensemble Sampler 506, Monte Carlo dropout sampler 508 and Batch Ensemble Sampler 510. Measuring the uncertainty of the NN predictions is essential for on-the-fly training. Since, if the uncertainty is higher than the user-defined threshold, then there is a need for retraining of the model, wherein the retraining can be fine-tuning with new data or adding more data and training the model again. Furthermore, the uncertainty block in the workflow calls for more quantum mechanical calculation and retraining of the NN model. There are various methods incorporated into the NN model to account for the uncertainty in the predictions such as Bayesian NN (BNN), Deep Ensemble, and Monte-Carlo (MC) dropout. The Bayesian NN (BNN) is one of the most popular methods for quantifying uncertainty by inferring predictive distribution. The variance in the predictive distribution is used to measure the uncertainty. Therefore, instead of having a single set of weights, weights and outputs are treated as probabilistic variables and we try to find the marginal distributions that best fit the data. Furthermore, the BNN would find the distributions of the weights by catering to the probability distributions. However, scaling BNN to a larger network is difficult. In addition, the Deep ensemble 506 is yet another powerful approach to measure uncertainty, where many copies of the model itself are trained on the same dataset. During inference, the average of the predictions or a combined output is treated as the prediction. Therefore, the variance or the distribution of the predictions is used to measure the uncertainty. However, the method is compute-intensive and storage-intensive. Furthermore, the Monte-Carlo (MC) dropout 508 is yet another method that is less compute-intensive to quantify uncertainty. MC dropout is a popular regularization technique to tackle over-fitting. Neurons are randomly deactivated, usually according to a Bernoulli distribution. In MC-dropout, such randomly sampled random subnetworks are then trained on the same data. Furthermore, the MC dropouts are applied at both training and during inference, and the inference is done multiple times, with different parameters being dropped in each iteration. Finally, the outputs are combined typically by averaging over the number of iterations to output the probability. Hence, MC dropout is a scalable way to learn a predictive distribution.

FIG. 5B illustrates a schematic workflow for uncertainty monitoring service, according to an embodiment herein. The prediction of the NNFF model is closely monitored through an uncertainty monitoring service 500. FIG. 5B, illustrates the workflow of the uncertainty monitoring service 500. If the uncertainty prediction of the NNFF model crosses the user-specified uncertainty threshold 552, retraining is flagged, which means further steps would be simulated using AIMD first-principle-based methods, thus launching MD simulations and replenishing data store 554. Hence, the data generated in the form of typical trajectories are then used to retrain/fine-tune the NN model again. Hence, the workflow is H continued until the desired number of steps is simulated.

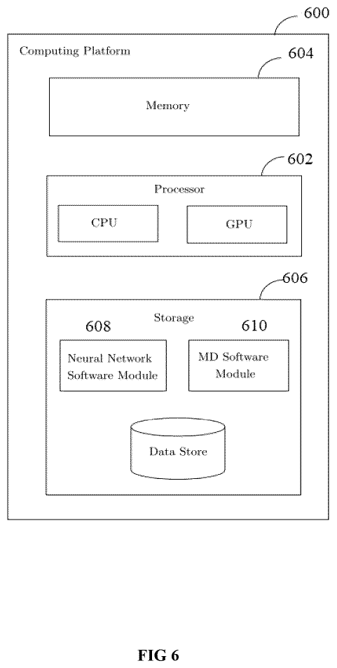

FIG. 6 illustrates a schematic diagram of a computing platform utilized to implement NNFF algorithm, according to one embodiment herein. FIG. 6 illustrates the computing platform 600 including a processor 602, memory 604, and non-volatile storage 606. The processor 602 includes one or more devices selected from high-performance computing (HPC) systems including high-performance cores, microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on computer-executable instructions residing in memory 604. The memory 604 includes a single memory device or a number of memory devices including, but not limited to, random access memory (RAM), volatile memory, non-volatile memory, static random-access memory (SRAM), dynamic random-access memory (DRAM), flash memory, cache memory, or any other device capable of storing information. The non-volatile storage 606 includes one or more persistent data storage devices such as a hard drive, optical drive, tape drive, non-volatile solid-state device, cloud storage, or any other device capable of persistently storing information. The processor 602 is configured to read into memory 604 and execute computer-executable instructions residing in Neural Network software module 608 of the non-volatile storage 606. Furthermore, processor 602 is further configured to read into memory 604 and execute computer-executable instructions residing in MD software module 610 of the non-volatile storage 606. The software modules 608 and 610 includes operating systems and applications. In addition, upon execution by the processor 602, the computer-executable instructions of the NNFF software module 608 and the MD software module 610 cause the computing platform 600 to implement one or more of the NNFF algorithms and/or methodologies and MD algorithms and/or methodologies, respectively.

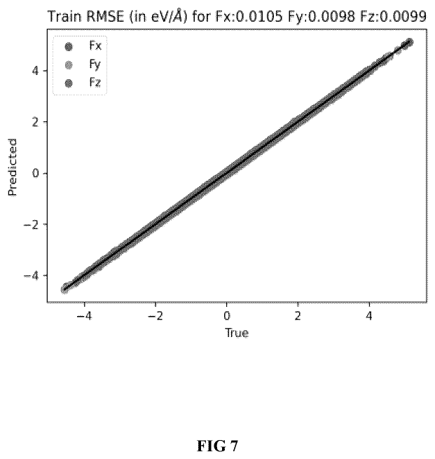

FIG. 7 illustrates depicts graph showing results for the prediction error in forces during molecular simulation of the lithium thiophosphate (LiPS) system, according to an embodiment herein. FIG. 7 illustrates the prediction error in forces during molecular simulation of lithium thiophosphate (LiPS) system. The prediction error between NNFF algorithm and the true value is Fₓ: 0.0105 eV/A₀, F_{y}: 0.0098 eV/A⁰ and F_{z}: 0.0099 eV/A ° respectively for the prediction of lithium thiophosphate, which is within the acceptable range.

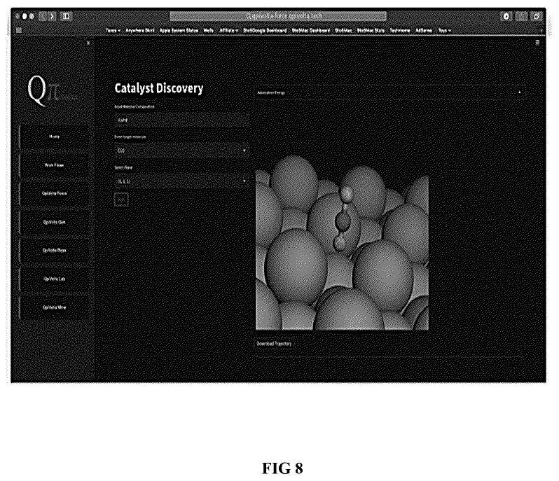

FIG. 8 illustrates a screenshot of the user interface for performing molecular dynamics simulations of catalyst using NNFF algorithm, according to an embodiment herein. The FIG. 8 illustrates a screenshot of the user interface, performing molecular dynamics simulations of a catalyst using NNFF algorithm.

It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

While the disclosure is susceptible to various modifications and alternative forms, specific embodiment thereof has been shown by way of example in the drawings and will be described in detail above. It should be understood, however that it is not intended to limit the disclosure to the forms disclosed, but on the contrary, the disclosure is to cover all modifications, equivalents, and alternatives falling within the scope of the disclosure.

### Advantages of the Embodiments Herein

The embodiments herein disclose a system and method for performing accelerated molecular dynamics computer simulations with an uncertainty-aware neural network. The embodiments herein provide a system and method to couple uncertainty monitored NNs with molecular dynamics for directly predicting the energy of the system and forces acting on atoms in molecular dynamic simulation of materials, polymers, and molecules systems with application in, but not limited to, electrochemical, photoelectrochemical and semiconductor devices. The embodiments herein work by coupling NNs with AIMD for on-the-fly training and prediction of interatomic potentials. Forces are then computed by taking the energy gradient with respect to positions. The NN prediction accelerates the quantum simulation by directly predicting the energy instead of self-consistent quantum calculation. Furthermore, the NN model is trained for the initial few frames of the trajectories on-the-fly from AIMD calculation. Subsequent frames of the trajectories is then predicted by the NN. Then, the trained model is used to run multiple AIMD simulations with random initialization for certainty. Hence, training the NN model on each material helps the model generalize to that specific material and learn the atom embedding from lesser data.

Moreover, the NNs and ML are used in the development of force fields, developing exchange-correlation functionals for DFT, predicting the structure of novel materials, calculating material properties, optimizing processes with active learning, and many more. The embodiments herein, though, are explained for the development of force fields on the fly or for accelerating simulations, can be integrated into other workflows as well. In other embodiments, similar workflows are used to predict molecular properties like adsorption energies, ionic conductivity, electronic conductivity, etc. Furthermore, a major step of an ML algorithm for any application is to represent the material data as features. It has to distinguish between different materials and encode relevant information, such as structure and composition. The extent of feature engineering depends on the algorithm itself. For example, modern architecture such as graph NNs and other geometric deep learning is part of the model itself. An ideal representation takes into account the invariance in symmetries, which includes rotation, reflection, translation, and permutation. Hence, the present invention can be integrated into any architecture with a few hidden layers. In the case of architectures without hidden layers, uncertainty can be monitored with some of the listed methods. According to different embodiments, graph NNs, transformers, equivariant NNs, and diffusion models may be used. Therefore, the ML models can be coupled with small high-fidelity calculations and monitored on-the-fly for uncertainty. Such hybrid models can provide high accuracy while maintaining the large speedup of ML. Furthermore, the hybrid models allow us to probe phenomena at longer time scales and larger length scales with high accuracy.

Although the embodiments herein are described with various specific embodiments, it will be obvious for a person skilled in the art to practice the embodiments herein with modifications.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments.

It is to be understood that the phrases or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

What is claimed is:

1. A method (100) for performing accelerated molecular dynamics computer simulations with an uncertainty-aware neural network, the method comprising:
a. receiving an input request to initialize simulations, comprising initial material 3D structure or compositions, and metadata comprising of parameters relevant for MD or molecular dynamics simulations (102);
b. running the molecular dynamics simulations based on the input request, to predict the simulation trajectory of movement of atoms, comprising energy of the system and forces acting on the atoms as simulation (104);
c. storing the simulation trajectories in a data store (106);
d. training a neural network force field NNFF using the simulation trajectories stored in the data store (108);
e. storing the trained NNFF parameters in a model store (110);
f. running the molecular dynamics simulations with the trained NNFF parameters stored in the model store to predict the further steps of simulation trajectory movement of atoms, comprising energy of the system and forces acting on the atoms as simulation (112);
g. monitoring uncertainty involved while predicting further simulation trajectory of movement of atoms by the trained NNFF (114);
h. launching MD simulations and replenishing the data store for training or retraining the NNFF, in case of detecting uncertainty over the threshold (116); and
i. calculating material properties by post-processing of the MD simulation trajectory data (118).

2. The method (100) according to claim 1, wherein the input request to initialize simulations comprises atom types, a 3D structure including coordinates, lattice parameters, the composition of the system, and the velocity of the atoms.

3. The method (100) according to claim 1, wherein the metadata parameters comprise the values corresponding to ensembles, including NVT, number of particles (N), volume (V), temperature (T), NPT, number of particles (N), pressure (P), temperature (T), and NVE, number of particles (N), volume (V), energy (E), integration algorithm to numerically solve the equations of motion, timestep to determine the frequency of integration of equations of motion, boundary conditions, and cutoff distance.

4. The method (100) according to claim 1, wherein the molecular dynamics simulations based on the input request, to predict the simulation trajectory of the movement of atoms is run for a number of timesteps by MD worker nodes; and wherein the number of timesteps depends upon the complexity of the system, including large and small systems, and wherein the large system involves more interactions and longer equilibrium times, and the small system involves fewer interactions and shorter equilibrium times.

5. The method (100) according to claim 1, wherein the method for training neural network force field NNFF comprises the steps of:
a. using the simulation trajectories stored in the datastore generated based on MD simulations; and wherein the simulation trajectories include snapshots of the system at different timesteps;
b. normalizing and preprocessing the simulation trajectories;
c. defining loss function that quantifies the error between the predicted future steps and the actual future steps in the simulation; and
d. training the NNFF using the prepared simulation trajectories and the loss function.

6. The method (100) according to claim 1, wherein the neural network force field NNFF is trained using the simulation trajectories stored in the data store for a number of iterations by NN trainer nodes; and wherein the number of iterations is a hyperparameter, which is set to a default value by a user, and changed accordingly depending on the system needs to be simulated.

7. The method (100) according to claim 1, wherein the uncertainty is monitored by an uncertainty monitoring service while running further MD with the NNFF; and wherein the uncertainty monitoring service detects uncertainty above prescribed threshold the method (100) involves launching MD simulations and replenishing the data store for training or retraining the NNFF.

8. The method (100) according to claim 7, wherein the training or retraining involves fine-tuning with new data or adding more data and training the NNFF module again with the inputs from molecular dynamics.

9. The method (100) according to claim 1, wherein the post-processing of MD simulation trajectory data involves analyzing and extracting various properties and insights from the simulated atomic trajectories, and the properties provide valuable information about the behavior of the system under study; and wherein the properties include structural properties, thermodynamic properties, dynamics and kinetics, chemical properties, hydration properties, electrostatic and non-bonded interactions, conformational analysis, hydrogen bond analysis, energetics, and other associated properties.

10. A system (200) for performing accelerated molecular dynamics computer simulations with an uncertainty-aware neural network, the system comprising:
a. an input module (202) configured to receive an input request to initialize simulations, comprising initial material 3D structure or compositions, and metadata comprising of parameters relevant for MD or molecular dynamics simulations;
b. a molecular dynamics (MD) module (204) configured to receive the input request from the input module and run the molecular dynamics simulations based on the input request, to predict the simulation trajectory of movement of atoms, comprising energy of the system and forces acting on the atoms as simulation, and storing the simulation trajectories in a data store;
c. a neural network force field NNFF module (206) configured to receive the simulation trajectory of movement of atoms from the MD module and train the network force field NNFF module using the simulation trajectories stored in the data store, and store the trained NNFF parameters in a model store; and wherein the NNFF module is further configured to run the molecular dynamics simulations with the trained NNFF parameters stored in the model store to predict the further steps of simulation trajectory movement of atoms, comprising energy of the system and forces acting on the atoms as simulation; and
d. an uncertainty monitoring service module (208) configured to monitor uncertainty involved while predicting further simulation trajectory of movement of atoms by the NNFF module, and also configured to request more data from the MD module to replenish the data store for training or retraining the NNFF module again in case of uncertainty over the threshold, and to calculate material properties by post-processing the MD simulation trajectory data.

11. The system (200) according to claim 10, wherein the input request by the input module to initialize simulations comprises atom types, a 3D structure including coordinates, lattice parameters, the composition of the system, and the velocity of the atoms.

12. The system (200) according to claim 10, wherein the metadata parameters comprise the values corresponding to ensembles, including NVT, number of particles (N), volume (V), temperature (T), NPT, number of particles (N), pressure (P), temperature (T), and NVE, number of particles (N), volume (V), energy (E), integration algorithm to numerically solve the equations of motion, timestep to determine the frequency of integration of equations of motion, boundary conditions, and cutoff distance.

13. The system (200) according to claim 10, wherein the molecular dynamics simulations based on the input request by the MD module, to predict the simulation trajectory of movement of atoms is run for a number of timesteps by MD worker nodes; and wherein the number of timesteps depend upon the complexity of the system, including large and small system, and wherein the large system involves more interactions and longer equilibrium times, and the small system involves fewer interactions and shorter equilibrium times.

14. The system (200) according to claim 10, wherein the method for training neural network force field NNFF in the NNFF module comprises the steps of:
a. using the simulation trajectories stored in the datastore generated based on MD simulations; and wherein the simulation trajectories include snapshots of the system at different timesteps;
b. normalizing and preprocessing the simulation trajectories;
c. defining loss function that quantifies the error between the predicted future steps and the actual future steps in the simulation; and
d. training the NNFF using the prepared simulation trajectories and the loss function.

15. The system (200) according to claim 10, wherein the neural network force field NNFF module is trained using the simulation trajectories stored in the data store for a number of iterations by NN trainer nodes; and wherein the number of iterations is a hyperparameter, which is set to a default value by a user, and changed accordingly depending on the system needs to be simulated.

16. The system (200) according to claim 10, wherein the uncertainty monitoring service module is configured to monitor uncertainty by incorporating uncertainty quantification into the NNFF module, provided if the uncertainty detected is above prescribed threshold, then the uncertainty monitoring service module is configured to requests more data from the MD module and replenishing the data store for training or retraining the NNFF.

17. The system (200) according to claim 16, wherein the training or retraining involves fine-tuning with new data or adding more data and training the NNFF module again with the inputs from the molecular dynamics module; and wherein the uncertainty quantification helps in making more informed decisions and assessing the reliability of the model predictions.

18. The system (200) according to claim 10, wherein the post-processing of MD simulation trajectory data involves analyzing and extracting various properties and insights from the simulated atomic trajectories, and the properties provide valuable information about the behavior of the system under study; and wherein the properties include structural properties, thermodynamic properties, dynamics and kinetics, chemical properties, hydration properties, electrostatic and non-bonded interactions, conformational analysis, hydrogen bond analysis, energetics, and other associated properties.
